(19) United States
(12) Patent Application Publication
(10) Pub. No.: US 2014/0236176 A1
(43) Pub. Date: Aug. 21, 2014

(54) Method for mapping sensor signals to output channels for neural activation

(71) Applicants: RANU JUNG, MIAMI, FL (US); JAMES J. ABBAS, SCOTTSDALE, AZ (US); BRIAN P. SMITH, DUBUQUE, IA (US); KENNETH HORCH, FOUNTAIN HILLS, AZ (US)

(72) Inventors: RANU JUNG, MIAMI, FL (US); JAMES J. ABBAS, SCOTTSDALE, AZ (US); BRIAN P. SMITH, DUBUQUE, IA (US); KENNETH HORCH, FOUNTAIN HILLS, AZ (US)

(21) Appl. No.: 14/181,172

(22) Filed: Feb. 14, 2014

Related U.S. Application Data
(60) Provisional application No. 61/765,565, filed on Feb. 15, 2013.

(51) Int. Cl. A61N 1/372; A61B 19/00

(52) U.S. Cl. CPC A61N 1/37264; A61B 19/2203; A61B 2019/2207
USPC 606/130; 607/62

(57) ABSTRACT

A method for mapping sensor signals to stimulation values and a device using the same are disclosed. The method includes the steps of receiving sensor signal from sensors, mapping the sensor signals to stimulation values, and delivering stimulation signals based on the stimulation values. The mapping may be performed using a linear, piecewise linear, or non-linear function. The method may be incorporated in an advanced prosthetic system that activates sensory neurons in a residual limb to provide an amputee with sensations related to grip force and hand opening. The method for implementing sensory feedback may also be incorporated into mechanical or robotic devices such as those used for surgery, games, and/or tele-manipulation.

### CROSS-REFERENCE TO RELATED APPLICATION

The present application claims the benefit of U.S. Provisional Application Ser. No. 61/765,565, filed Feb. 15, 2013, which is hereby incorporated by reference herein in its entirety, including any figures, tables, and drawings.

This invention was made with government support under contract number EB008578 awarded by the National Institutes of Health. The government has certain rights in the invention.

### BACKGROUND

Short pulses of electrical stimulation can be used to produce action potentials in neurons. If sensory neurons are stimulated, they can elicit sensations. For example, stimulating neurons in the cochlea of the ear can elicit the sensation of sound, stimulating neurons in the retina of the eye can elicit sensations of light, and stimulating sensory neurons emanating from the fingertips can elicit the sensation of touch. Similarly, if motor neurons are stimulated, they can elicit muscle contractions.

### BRIEF SUMMARY

Embodiments of the subject invention relate to methods and devices mapping sensor signals to stimulation values. In an embodiment, a method of mapping sensor signals to stimulation values comprises: receiving sensor signals from a plurality of sensors; mapping the sensor signals to stimulation values; and delivering stimulation signals according to the stimulation values.

The mapping step may use a piecewise linear function.

The piecewise linear function may have a threshold value below which no stimulation is sent and a saturation value above which stimulation is delivered at a maximum level.

In a further embodiment, the method may include a step of calibrating the threshold and saturation values to a user. The sensor signals between the threshold and saturation values may be scaled proportionally.

The stimulation values may represent pulse frequency.

The mapping step may utilize a linear function or a non-linear function.

The mapping step may utilize dynamic mapping, in which the current values of the outputs of the mapping function are derived from the current values of the inputs and the past history of inputs to the mapping function.

In an embodiment, a method of mapping sensor signals to stimulation pulses comprises the steps of: detecting sensor signals from a plurality of sensor channels; calculating stimulation values based on the sensor signals; and delivering stimulation pulses based on the stimulation values on a plurality of output channels.

The step of calculating stimulation values may use a piecewise linear function having a threshold value below which no stimulation is sent and a saturation value above which stimulation is delivered at a maximum level, though embodiments are not limited thereto.

The step of calculating stimulation values may use a dynamic mapping function, in which the current values of the outputs of the mapping function are derived from the current values of the inputs and past history of inputs to the mapping function.

The step of calculating stimulation values may use a non-linear function.

A single sensor channel may be used to calculate stimulation values on more than one output channel.

More than one sensor channel may be used to calculate stimulation values on one output channel.

The stimulation values may represent, for example, pulse frequency, pulse amplitude, pulse width, or any other parameter value that may alter pulse shape.

The stimulation pulses may be provided to electrodes implanted in a residual limb.

In an embodiment of the present invention, an apparatus comprises: at least one sensor for producing a sensor signal representative of a sensed characteristic; a processor configured to receive the sensor signal and convert the sensor signal to a stimulation value representative of a desired amount of stimulation; and a stimulation pulse generator for delivering stimulation based on the stimulation value. The apparatus can be, for example, a prosthetic apparatus.

The processor may implement a piecewise linear function, and the piecewise linear function may have a threshold value below which no stimulation is sent and a saturation value above which stimulation is delivered at a maximum level, though embodiments are not limited thereto.

The step of calculating stimulation values may use a dynamic mapping function or a non-linear function.

A single sensor channel may be used to calculate stimulation values on more than one output channel. More than one sensor channel may be used to calculate stimulation values on an output channel.

The stimulation values may represent, for example, pulse frequency. The stimulation values may represent pulse intensity by specifying, e.g., pulse amplitude, pulse duration, or any other parameter that affects pulse shape.

The stimulation pulses may be provided to electrodes on the surface of the skin or implanted in the body.

In an embodiment of the present invention, a mechanical or robotic tool (e.g., those used for surgery, games, and/or tele-manipulation) may comprise: at least one sensor for producing a sensor signal representative of a sensed characteristic; a processor configured to receive the sensor signal and convert the sensor signal to a stimulation value representative of a desired amount of stimulation; and a stimulation pulse generator for delivering stimulation based on the stimulation value.

The processor may implement a piecewise linear function, and the piecewise linear function may have a threshold value below which minimal or no stimulation is sent and a saturation value above which stimulation is delivered at a maximum level, though embodiments are not limited thereto.

The step of calculating stimulation values may use a dynamic mapping function or a non-linear function.

A single sensor channel may be used to calculate stimulation values on more than one output channel. More than one sensor channel may be used to calculate stimulation values on an output channel.

The stimulation values may represent, for example, pulse frequency. The stimulation values may represent pulse intensity by specifying, e.g., pulse amplitude, pulse duration, or any other parameter that affects pulse shape.

The stimulation pulses may be provided to electrodes on the surface of the skin or implanted in the body.

### BRIEF DESCRIPTION OF DRAWINGS

The following drawings form part of the present specification and are included to further demonstrate certain aspects of the present invention. The invention may be better understood by reference to the drawings in combination with the detailed description of specific embodiments presented herein.

FIG. 1 is a block diagram of a method according to an embodiment of the present invention.

FIG. 2 is a chart showing a piecewise linear function according to an embodiment of the present invention.

FIG. 3 is a block diagram of a prosthetic according to an embodiment of the present invention.

FIG. 4 is a block diagram of an ANS-NEP neural stimulation system according to an embodiment of the present invention.

### DETAILED DISCLOSURE

In the following detailed description, reference is made to the accompanying drawings, in which are shown exemplary but non-limiting and non-exhaustive embodiments of the invention. These embodiments are described in sufficient detail to enable those having skill in the art to practice the invention, and it is understood that other embodiments may be used, and other changes may be made, without departing from the spirit or scope of the invention. The following detailed description is, therefore, not to be taken in a limiting sense, and the scope of the invention is defined only by the appended claims.

For systems that are designed to convey sensory information to the user by activating afferent neurons, it can be desirable to gather information from several sensors and to use that information to determine the stimulation levels on each of several channels of sensory stimulation. For systems that are designed to control processes by activating neuromuscular tissue, it can be desirable to gather information from several sensors and to use that information to determine the stimulation levels on each of several channels of motor neuron stimulation. In either of these scenarios, it would be advantageous to have a method for appropriately and efficiently mapping the set of sensor signals to the set of outputs.

Short pulses of electrical stimulation can be used to produce action potentials in neurons. When such stimulation pulses are used to activate sensory neurons, they can elicit sensations; if the stimulation is linked to signals derived from external sensors, a person will experience sensations that are synchronized with the measured signals. In many of the possible applications that use electrical stimulation, it is desirable to use information from a set of sensors to determine stimulation levels on a set of stimulation channels.

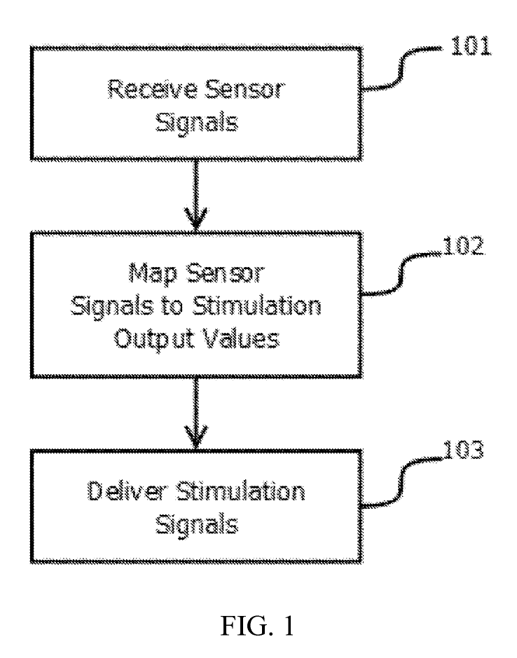

For example, in a prosthetic limb, information from a set of signals from transducers may be used to produce a set of pulse trains on stimulation electrodes. Referring to FIG. 1, in step 101 one or more sensor signals are received from a set of sensors. In step 102, the sensor signals (e.g., a set of sensor signals) are mapped to a set of stimulation output values. In one embodiment, the stimulation output values include stimulation frequency. In step 103, stimulation signals are delivered based on the stimulation output values.

In step 101, a set of sensors mounted on a device (e.g., mechanical device such as a prosthetic limb) can be used to produce sensor signals. If analog sensors are used, the sensor signals are digitized at regular time intervals. If digital sensors are used, no analog conversion is necessary. Standard low-pass or high-pass filtering on the time-series values from each of the sensors can be utilized to condition the signals, for example to reduce high frequency noise, remove DC and low-frequency signals, differentiate the signal, integrate the signal, etc. In an exemplary embodiment, four sensors are utilized in a prosthetic arm. These sensors produce values representative of, for example, normal force, lateral force, axial force, and position.

In step 102, the sensor signals can be mapped to output stimulation values using an algorithm. For convenience, the algorithm will be referred to as a Sensor Stimulation Mapping (SSM) algorithm, though embodiments are not limited thereto. The SSM algorithm calculates pulse parameters for all the stimulation channels based on the sensor values and user specific stimulation settings (e.g., pulse amplitude, pulse width, minimum pulse frequency, maximum pulse frequency). The user specific stimulation settings can be determined during a calibration process and can be stored in memory that is accessible by the SSM algorithm. The pulse parameters can be restricted to be within the minimum and maximum values determined during a calibration process. At each time increment, the algorithm can receive sensor signals (e.g., four sensor signals) as inputs. The sensor signals can be in the form of digitized input. A linear mapping between sensor input and stimulation output can be performed according to the following equations:

X=G_{S} (1)

PFi=GPF i(Xi−Xmin i)+PFmin i if X min i:S Xi:S Xmax I (2)

PFi=0 if Xi<Xmin_{—}i (3)

PFi=PFmax I if Xi>Xmax i (4)

In equations (1)-(4), each AD (analog to digital) value represents an offset-adjusted AD value taken from a sensor. Sensor gain (Gs) represents a matrix containing gain values applied to each sensor for a given stimulation channel. PFi represents the final calculated pulse frequency for channel ‘i’, while Xi represents an intermediary value for that channel based on the sensor gains and sensor values. Xmin_i and Xmax_i represent the range over which Xi values are to be converted directly to PFi values. PFmin_i and PFmax_i represent the minimum and maximum pulse frequency values used on a given channel, which are determined during the calibration process.

The stimulation system may be designed to accept pulse period values as inputs, in which case the calculated pulse frequency values are converted to corresponding pulse periods, e.g. via a look-up table. The use of a look-up table minimizes computation time and firmware complexity.

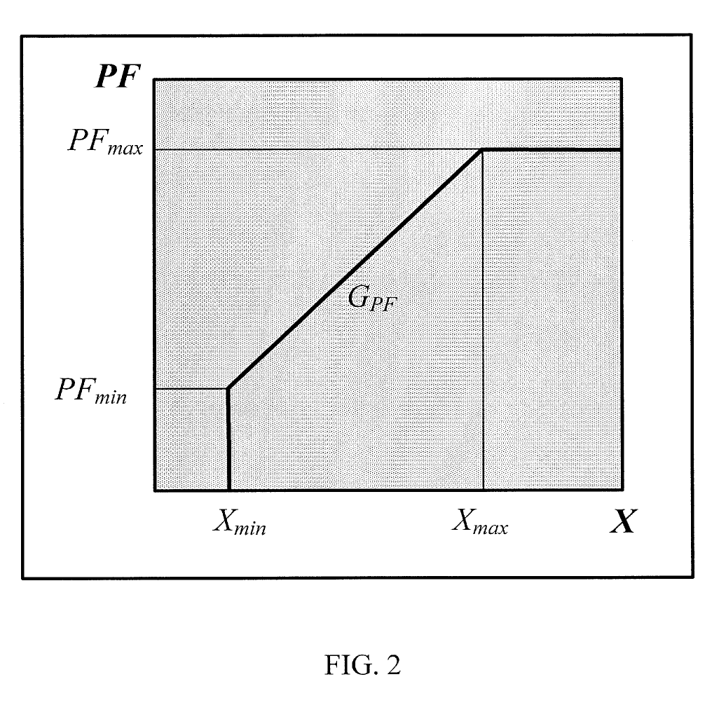

The piecewise linear function uses a threshold below which no stimulation, or a pre-determined minimum level of stimulation, is sent and a saturation value above which stimulation is delivered at the maximum level. Inputs between the threshold and saturation values are scaled proportionally. FIG. 2 illustrates a piecewise linear function.

The number of input channels may be increased by creating new channels as processed versions of and/or combinations of the original set of sensor channels. F or example, a new input channel can be created by differentiating one of the channels or by combining the processed values from two or more input channels.

The outputs from the SSM algorithm are a set of numbers that indicate the level of stimulation to be produced on each of several channels. The specification of ‘level’ will depend on the type of stimulation being used and on the manner in which stimulation intensity is being varied. For example, if electrical stimulation is being used, then pulse parameters such as pulse amplitude and pulse width could be set at fixed values and the output of the SSM algorithm could be the value for instantaneous pulse frequency on that channel. In this way, the SSM algorithm would use the set of sensor signals to modulate intensity of sensations by modulating pulse frequencies of each of a number of output channels. At each time step, the SSM algorithm can map the set of (conditioned) sensor signals to a set of values for stimulation across a set of multiplexed output channels.

The SSM algorithm may be implemented on a specialized DSP.

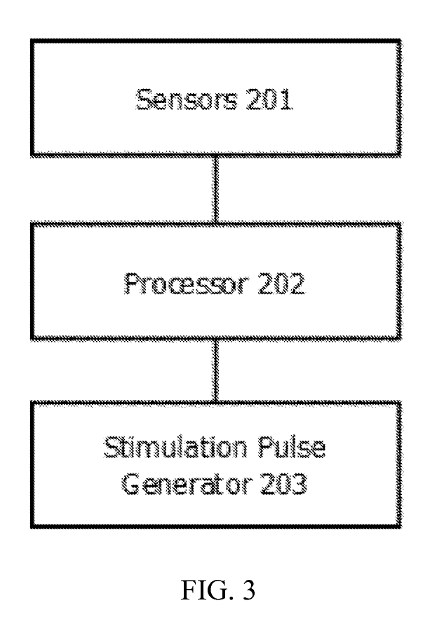

Referring to FIG. 3, in an embodiment, the SSM algorithm is embodied in a device (e.g., an advanced prosthetic system) that activates sensory neurons in a residual limb to provide an amputee with sensations related to grip force and hand opening. In this system, information derived from sensors 201 in the hand is used by a processor 202 to modulate the frequency of stimulation pulse trains generated by a stimulation pulse generator 203 on each of several electrodes implanted in the residual limb. This can provide the user with multiple graded sensations that reflect the state of the prosthetic limb.

The SSM algorithm can be useful in any neural stimulation system that benefits from using input from multiple sensors and can be particularly useful in systems that use multiple output channels and those that require real-time operation. In addition to its use in stimulation systems, the method would be useful in situations where multiple sensor signals are used to determine the outputs on multiple channels and where the mapping of sensor reading to output is linear, piecewise-linear, or non-linear.

Although the SSM algorithm has been described in the context of a prosthetic limb, it can be used in conjunction with any type of robotic tool, such as those used for surgery, games, and/or tele-manipulation.

To elicit sensation in the user, a method of the subject invention can utilize any form of stimulation, including but not limited to electrical, optical, magnetic, vibratory, and/or tactile.

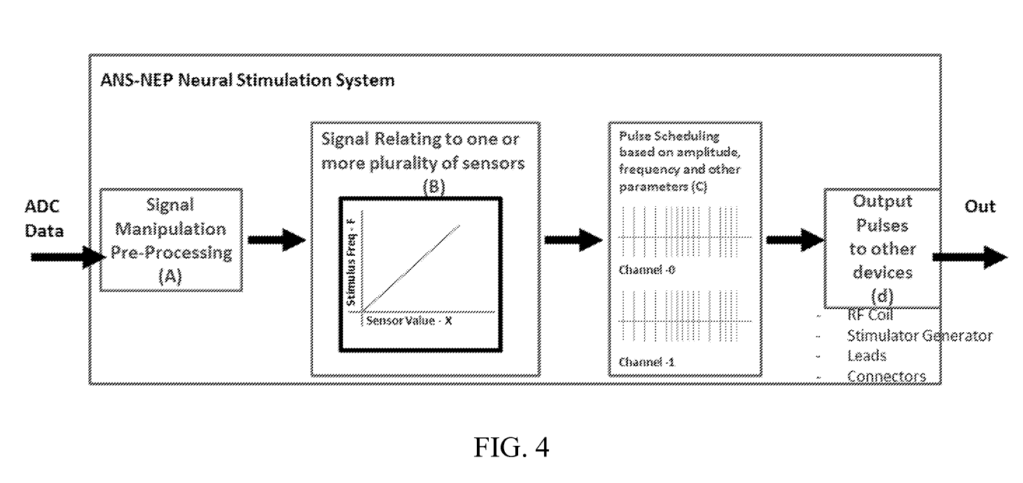

FIG. 4 is a block diagram of an ANS-NEP neural stimulation system according to an embodiment of the present invention. Referring to FIG. 4, a system can receive ADS data and perform signal manipulation pre-processing A. Then, the signal can be related to one or more sensors B, and pulse scheduling C can be performed, based on, e.g., amplitude, frequency, and/or other factors. Output pulses D can be sent to other devices, including but not limited to connectors, leads, stimulator generators, and/or RF coils.

The disclosed methods and systems provide several advantages, including but not limited to: allowing the simultaneous use of several sensors (inputs) to determine levels on several stimulation channels (outputs) to produce a set of meaningful sensations; providing the ability to reduce the effects of cross-talk on sensor input lines; providing the ability to use processed sensor inputs to determine stimulation levels; providing the ability to combine information on several sensor input channels to determine stimulation level on one channel; providing the ability to use one sensor input channel to derive stimulation levels for several output channels; and providing the ability to utilize a piecewise linear or nonlinear mapping function to determine stimulation levels from a set of processed sensor inputs.

In the embodiment described above, the SSM algorithm utilizes a piecewise linear function. However, a linear or a nonlinear mapping function can also be used. Alternatively, the SSM algorithm can utilize dynamic mapping (e.g., one in which the previous history of sensor signals influences the current stimulation values or one in which current sensor signals influence the current stimulation value and future values).

The SSM algorithm can utilize a single sensor channel to specify different stimulation values on various output channels. The SSM algorithm can utilize more than one sensor input to influence the resulting stimulation on any given output channel.

### Exemplified Embodiments

The invention includes, but is not limited to, the following embodiments:

### Embodiment 1

A method of mapping sensor signals to stimulation values, comprising:
receiving sensor signals from a plurality of sensors;
mapping the sensor signals to stimulation values; and
delivering stimulation signals according to the stimulation values.

### Embodiment 2

The method according to embodiment 1, wherein the mapping step utilizes a piecewise linear function.

### Embodiment 3

The method according to embodiment 2, wherein the piecewise linear function has a threshold value below which minimal or no stimulation is sent and a saturation value above which stimulation is delivered at a maximum level.

### Embodiment 4

The method according to embodiment 3, further comprising calibrating the threshold and saturation values.

### Embodiment 5

The method according to any of embodiments 3-4, wherein sensor signals between the threshold and saturation values are scaled proportionally.

### Embodiment 6

The method of any of embodiments 1-5, wherein the stimulation values represent pulse frequency.

### Embodiment 7

The method of any of embodiments 1 or 6, wherein the mapping step utilizes a linear function.

### Embodiment 8

The method of any of embodiments 1 or 6, wherein the mapping step utilizes a non-linear function.

### Embodiment 9

The method of any of embodiments 1 or 6, wherein the mapping step utilizes dynamic mapping.

### Embodiment 10

A method of mapping sensor signals to stimulation pulses, comprising the steps of:
detecting sensor signals from a plurality of sensor channels;
calculating stimulation values based on the sensor signals; and
delivering stimulation pulses based on the stimulation values on a plurality of output channels.

### Embodiment 11

The method of embodiment 10, wherein the step of calculating stimulation values uses a piecewise linear function.

### Embodiment 12

The method of embodiment 11, wherein the piecewise linear function has a threshold value below which minimal or no stimulation is sent and a saturation value above which stimulation is delivered at a maximum level.

### Embodiment 13

The method of embodiment 10, wherein the step of calculating stimulation values uses a dynamic mapping function.

### Embodiment 14

The method of embodiment 10, wherein the step of calculating stimulation values uses a non-linear function.

### Embodiment 15

The method of any of embodiments 10-14, wherein a single sensor channel is used to calculate stimulation values on more than one output channel.

### Embodiment 16

The method of any of embodiments 10-14, wherein more than one sensor channel is used to calculate stimulation values on an output channel.

### Embodiment 17

The method of any of embodiments 10-16, wherein the stimulation values represent pulse frequency.

### Embodiment 18

The method of any of embodiments 10-16, wherein the stimulation values represent pulse intensity by specifying pulse amplitude, pulse duration, or any other parameter that affects pulse shape.

### Embodiment 19

The method of any of embodiments 10-18, wherein the stimulation pulses are provided to electrodes implanted in a residual limb.

### Embodiment 20

A prosthetic apparatus, comprising:
at least one sensor for producing a sensor signal representative of a sensed characteristic;
a processor configured to receive the sensor signal and convert the sensor signal to a stimulation value representative of a desired amount of stimulation; and
a stimulation pulse generator for delivering stimulation based on the stimulation value.

### Embodiment 21

The apparatus according to embodiment 20, wherein the processor implements a piecewise linear function.

### Embodiment 22

The apparatus according to embodiment 21, wherein the piecewise linear function has a threshold value below which minimal or no stimulation is sent and a saturation value above which stimulation is delivered at a maximum level.

### Embodiment 23

The apparatus according to embodiment 20, wherein the step of calculating stimulation values uses a dynamic mapping function.

### Embodiment 24

The apparatus according to embodiment 20, wherein the step of calculating stimulation values uses a non-linear function.

### Embodiment 25

The apparatus according to any of embodiments 20-24, wherein a single sensor channel is used to calculate stimulation values on more than one output channel.

### Embodiment 26

The apparatus according to any of embodiments 20-24, wherein more than one sensor channel is used to calculate stimulation values on an output channel.

### Embodiment 27

The apparatus according to any of embodiments 20-26, wherein the stimulation values represent pulse frequency.

### Embodiment 28

The apparatus according to any of embodiments 20-26, wherein the stimulation values represent pulse intensity by specifying pulse amplitude, pulse duration or any other parameter that affects pulse shape.

### Embodiment 29

The apparatus according to any of embodiments 20-28, wherein the stimulation pulses are provided to electrodes placed on the surface of the skin or implanted in a residual limb.

### Embodiment 30

A mechanical or robotic tool, such as those used for surgery, games, and/or tele-manipulation, comprising:
at least one sensor for producing a sensor signal representative of a sensed characteristic;
a processor configured to receive the sensor signal and convert the sensor signal to a stimulation value representative of a desired amount of stimulation; and
a stimulation pulse generator for delivering stimulation based on the stimulation value.

### Embodiment 31

The apparatus according to embodiment 30, wherein the processor implements a piecewise linear function.

### Embodiment 32

The apparatus according to embodiment 31, wherein the piecewise linear function has a threshold value below which minimal or no stimulation is sent and a saturation value above which stimulation is delivered at a maximum level.

### Embodiment 33

The apparatus according to embodiment 30, wherein the step of calculating stimulation values uses a dynamic mapping function.

### Embodiment 34

The apparatus according to embodiment 30, wherein the step of calculating stimulation values uses a non-linear function.

### Embodiment 35

The apparatus according to any of embodiments 30-34, wherein a single sensor channel is used to calculate stimulation values on more than one output channel.

### Embodiment 36

The apparatus according to any of embodiments 30-34, wherein more than one sensor channel is used to calculate stimulation values on an output channel.

### Embodiment 37

The apparatus according to any of embodiments 30-36, wherein the stimulation values represent pulse frequency.

### Embodiment 38

The apparatus according to any of embodiments 30-36, wherein the stimulation values represent pulse intensity by specifying pulse amplitude, pulse duration or any other parameter that affects pulse shape.

### Embodiment 39

The apparatus according to any of embodiments 30-38, wherein the stimulation pulses are provided to electrodes on the surface of the skin or implanted in the body.

### Embodiment 40

The method of any of embodiments 1-9, wherein a single sensor is used to calculate stimulation values on more than one output channel.

### Embodiment 41

The method of any of embodiments 1-9, wherein more than one sensor is used to calculate stimulation values on one output channel.

### Embodiment 42

The method of any of embodiments 1-9, wherein more than one sensor is used to calculate stimulation values more than one output channel.

### Embodiment 43

The apparatus of any of embodiments 20-24 and 27-29, wherein more than one sensor channel is used to calculate stimulation values more than one output channel.

### Embodiment 44

The apparatus of any of embodiments 30-34 and 37-39, wherein more than one sensor channel is used to calculate stimulation values more than one output channel.

### Embodiment 45

The method of any of embodiments 10-14 and 17-19, wherein more than one sensor channel is used to calculate stimulation values more than one output channel.

The methods and processes described herein can be embodied as code and/or data. The software code and data described herein can be stored on one or more computer readable media, which may include be any device or medium that can store code and/or data for use by a computer system. When a computer system reads and executes the code and/or data stored on a computer-readable medium, the computer system performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); or other media now known or later developed that is capable of storing computer-readable information/data. Computer-readable media should not be construed or interpreted to include any propagating signals.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein (including those in the “References” section) are incorporated by reference in their entirety, including all figures and tables, to the extent they are not inconsistent with the explicit teachings of this specification.

What is claimed is:

1. A method of mapping sensor signals to stimulation values, comprising:
receiving sensor signals from a plurality of sensors;
mapping the sensor signals to stimulation values; and
delivering stimulation signals according to the stimulation values.

2. The method according to claim 1, wherein the mapping step utilizes a piecewise linear function.

3. The method according to claim 2, wherein the piecewise linear function has a threshold value below which minimal or no stimulation is sent and a saturation value above which stimulation is delivered at a maximum level.

4. The method according to claim 3, wherein sensor signals between the threshold and saturation values are scaled proportionally.

5. The method of claim 1, wherein the mapping step utilizes a non-linear function.

6. The method of claim 1, wherein the mapping step utilizes dynamic mapping.

7. A method of mapping sensor signals to stimulation pulses, comprising the steps of:
detecting sensor signals from a plurality of sensor channels;
calculating stimulation values based on the sensor signals; and
delivering stimulation pulses based on the stimulation values on a plurality of output channels.

8. The method of claim 7, wherein the step of calculating stimulation values uses a piecewise linear function.

9. The method of claim 8, wherein the piecewise linear function has a threshold value below which minimal or no stimulation is sent and a saturation value above which stimulation is delivered at a maximum level.

10. The method of claim 7, wherein the step of calculating stimulation values uses a non-linear function.

11. The method of claim 7, wherein the step of calculating stimulation values uses a dynamic mapping function.

12. The method of claim 7, wherein more than one sensor channel is used to calculate stimulation values on one output channel.

13. An apparatus comprising:
at least one sensor for producing a sensor signal representative of a sensed characteristic;
a processor configured to receive the sensor signal and convert the sensor signal to a stimulation value representative of a desired amount of stimulation; and
a stimulation pulse generator for delivering stimulation based on the stimulation value.

14. The apparatus according to claim 13, wherein the processor implements a piecewise linear function.

15. The apparatus according to claim 14, wherein the piecewise linear function has a threshold value below which minimal or no stimulation is sent and a saturation value above which stimulation is delivered at a maximum level.

16. The apparatus according to claim 13, wherein the step of calculating stimulation values uses a non-linear function.

17. The apparatus according to claim 13, wherein the step of calculating stimulation values uses a dynamic mapping function.

18. The apparatus according to claim 13, wherein more than one sensor is used to calculate stimulation values on one output channel of the apparatus.

19. The apparatus according to claim 13, wherein the apparatus is a mechanical or robotic tool, such as those used for surgery, games, or tele-manipulation.

20. The apparatus according to claim 13, wherein the apparatus is a prosthetic apparatus.
